(19) United States
(12) Patent Application Publication
(10) Pub. No.: US 2020/0143703 A1
(43) Pub. Date: May 7, 2020

(54) FIXED-GAZE MOVEMENT TRAINING SYSTEMS WITH VISUAL FEEDBACK AND RELATED METHODS

(71) Applicant: Starkey Laboratories, Inc., Eden Prairie, MN (US)

(72) Inventors: David Alan Fabry, Eden Prairie, MN (US); Achintya Kumar Bhowmik, Cupertino, CA (US); Justin R. Burwinkel, Eden Prairie, MN (US); Jeffery Lee Crukley, Milton (CA); Amit Shahar, Hod HaSharon (IL)

(21) Appl. No.: 16/677,238

(22) Filed: Nov. 7, 2019

Related U.S. Application Data
(60) Provisional application No. 62/756,886, filed on Nov. 7, 2018.

(51) Int. Cl. G09B 19/00; A61B 3/11; A61B 5/00; A61B 3/113; A61B 5/11

(52) U.S. Cl. CPC G09B 19/003; A61B 3/112; A61B 5/4863; A61B 5/0077; G09B 21/009; A61B 5/1126; A61B 5/7405; A61B 5/742; A61B 2562/0219; A61B 3/113

(57) ABSTRACT

Embodiments herein relate to hearing assistance devices and related systems and methods for providing visual feedback to a subject undergoing fixed-gaze movement training. In an embodiment, method of providing vestibular therapy to a subject is included. The method can include prompting the subject to execute an exercise, the exercise comprising a predetermined movement while maintaining a fixed point of eye gaze, tracking the point of gaze of the subject's eyes using a camera, and generating data representing a measured deviation between the fixed point of eye gaze and the tracked point of gaze. Other embodiments are included herein.

This application claims the benefit of U.S. Provisional Application No. 62/756,886, filed Nov. 7, 2018, the content of which is herein incorporated by reference in its entirety.

### FIELD

Embodiments herein relate to hearing assistance devices and related systems and methods for providing visual feedback to a subject undergoing fixed-gaze movement training.

### BACKGROUND

Each year, millions of patients visit a physician with complaints of dizziness. It is the most common complaint of patients over the age of 75, but it can occur in patients of any age. Dizziness is a general term that can be used to describe more specific feelings of unsteadiness, wooziness (swimming feeling in head), lightheadedness, feelings of passing out, sensations of moving, vertigo (feeling of spinning), floating, swaying, tilting, and whirling. Dizziness can be due to an inner ear disorder, a side effect of medications, a sign of neck dysfunction, or it can be due to a more serious problem such as a neurological or cardiovascular problem.

Conditions and symptoms related to dizziness can include imbalance, vertigo, Meniere's syndrome, benign paroxysmal positional vertigo (BPPV), vestibular neuritis, neck-related dizziness and migraines.

One approach to treating dizziness, imbalance, vertigo, Meniere's syndrome, benign paroxysmal positional vertigo (BPPV), neck-related dizziness and migraines is to have the patient perform exercises that can include vestibular rehabilitation exercises. Vestibular rehabilitation exercises are designed to improve balance and reduce problems related to dizziness. Beyond dizziness and the related conditions described above, vestibular rehabilitation may be used to treat patients who have had a stroke or brain injury or who have a propensity to fall.

### SUMMARY

Embodiments herein relate to hearing assistance devices and related systems and methods for providing visual feedback to a subject undergoing fixed-gaze movement training. In an embodiment, method of providing vestibular therapy to a subject is included. The method can include prompting the subject to execute an exercise, the exercise comprising a predetermined movement while maintaining a fixed point of eye gaze, tracking the point of gaze of the subject's eyes using a camera, and generating data representing a measured deviation between the fixed point of eye gaze and the tracked point of gaze.

In an embodiment, a hearing assistance device is included. The hearing assistance device can include a control circuit and an IMU in electrical communication with the control circuit. The IMU can be disposed in a fixed position relative to a head of a subject wearing the hearing assistance device. The hearing assistance device can include a microphone in electrical communication with the control circuit, an electroacoustic transducer for generating sound in electrical communication with the control circuit, and a power supply circuit in electrical communication with the control circuit. The control circuit can be configured to initiate a prompt to a subject to execute an exercise, the exercise comprising a predetermined movement while maintaining a fixed point of eye gaze and detect execution of the exercise using data derived from the IMU.

In an embodiment, a system for providing vestibular training for a subject is included. The system can include a hearing assistance device including a control circuit and an IMU in electrical communication with the control circuit. The IMU can be disposed in a fixed position relative to a head of a subject wearing the hearing assistance device. The hearing assistance device can further include a microphone in electrical communication with the control circuit, an electroacoustic transducer for generating sound in electrical communication with the control circuit, a power supply circuit in electrical communication with the control circuit, and an external visual display device in wireless data communication with the hearing assistance device. The external visual display device can include a video display screen and a camera. The system can be configured to prompt the subject to execute an exercise, the exercise comprising a predetermined movement while maintaining a fixed point of eye gaze. The system can further be configured to track the point of gaze of the subject's eyes using data from the camera and generate data representing a measured deviation between the fixed point of eye gaze and the tracked point of gaze.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a partial cross-sectional view of ear anatomy.

FIG. 2 is a schematic view of a hearing assistance device in accordance with various embodiments herein.

FIG. 3 is a schematic view of various components of a hearing assistance device in accordance with various embodiments herein.

FIG. 4 is a schematic view of a hearing assistance device disposed within the ear of a subject in accordance with various embodiments herein.

FIG. 5 is a schematic view of data flow as part of a system in accordance with various embodiments herein.

FIG. 6 is a schematic side view of a subject wearing a hearing assistance device in accordance with various embodiments herein.

FIG. 7 is a schematic side view of a subject wearing a hearing assistance device and executing a fixed gaze exercise in accordance with various embodiments herein.

FIG. 8 is a schematic top view of a subject wearing hearing assistance devices in accordance with various embodiments herein.

FIG. 9 is a schematic top view of a subject wearing hearing assistance devices and executing a fixed gaze exercise in accordance with various embodiments herein.

FIG. 10 is a schematic view of a subject wearing a hearing assistance device and receiving visual feedback from an external visual display device in accordance with various embodiments herein.

FIG. 11 is a schematic frontal view of a subject wearing hearing assistance devices in accordance with various embodiments herein.

FIG. 12 is a schematic view of an external visual display device and elements of the visual display thereof.

FIG. 13 is a schematic view of an external visual display device and elements of the visual display thereof.

FIG. 14 is a schematic view of a system in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Exercises such as vestibular rehabilitation exercises can be useful for patients experiencing dizziness, imbalance, vertigo, Meniere's syndrome, benign paroxysmal positional vertigo (BPPV), neck-related dizziness and migraines, and the like. Tracking the subject's eyes during such exercises, and specifically during fixed-gaze exercises, can provide useful information. For example, such information can be used to provide feedback and/or guidance to the subject. Such information can also be used to inform a care provider of the health state or the patient and trends regarding the same. Such information can also be used to identify acute vestibular decompensation events.

Embodiments herein include hearing assistance devices and related systems and methods for guiding patients through vestibular movement training exercises, such as fixed-gaze training exercises. In some embodiments the device or system can track the subject's eyes and the direction of their gaze during the exercise. In some embodiments, visual feedback can also be provided to assist the subject in performing the exercises properly and to provide them feedback regarding how well they are maintaining a fixed gaze. In addition, embodiments herein can include evaluating eye movement during exercise movements, such as to identify notable eye movements such as nystagmus. It will be appreciated that there are numerous classifications of nystagmus. The nystagmus observed in an individual may be either typical or atypical given circumstances and the activity of the individual. In some embodiments, the nystagmus can include horizontal gaze nystagmus. In addition, embodiments herein can include aspects of initiation of exercises or prompting a subject to do the same. In addition, embodiments herein can include systems for remote care providers or exercise leaders to provide guidance to a plurality of subjects.

The term “hearing assistance device” as used herein shall refer to devices that can aid a person with impaired hearing. The term “hearing assistance device” shall also refer to devices that can produce optimized or processed sound for persons with normal hearing. Hearing assistance devices herein can include hearables (e.g., wearable earphones, headphones, earbuds, virtual reality headsets), hearing aids (e.g., hearing instruments), cochlear implants, and bone-conduction devices, for example. Hearing assistance devices include, but are not limited to, behind-the-ear (BTE), in-the ear (ITE), in-the-canal (ITC), invisible-in-canal (IIC), receiver-in-canal (RIC), receiver in-the-ear (RITE) or completely-in-the-canal (CIC) type hearing assistance devices or some combination of the above.

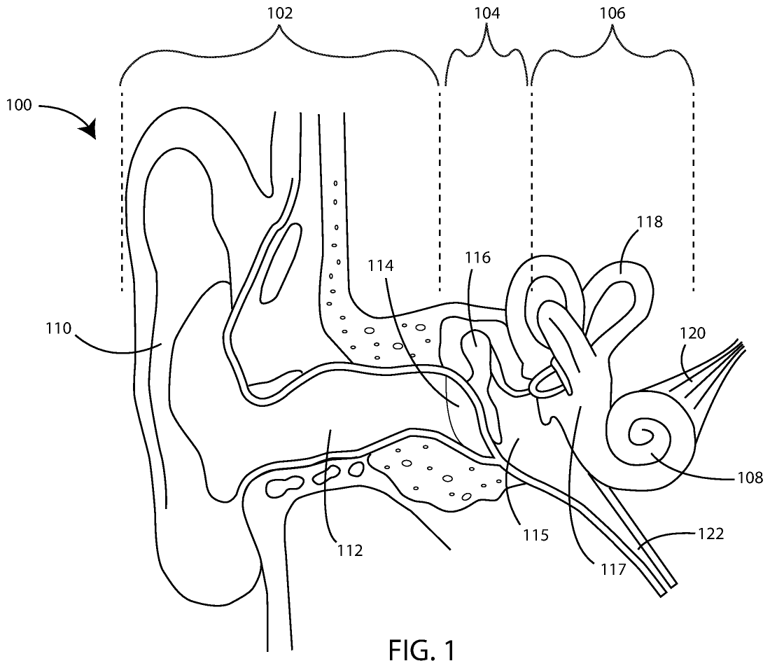

Referring now to FIG. 1, a partial cross-sectional view of ear anatomy 100 is shown. The three parts of the ear anatomy 100 are the outer ear 102, the middle ear 104 and the inner ear 106. The outer ear 102 includes the pinna 110, ear canal 112, and the tympanic membrane 114 (or eardrum). The middle ear 104 includes the tympanic cavity 115 and auditory bones 116 (malleus, incus, stapes). The inner ear 106 includes the cochlea 108, vestibule 117, semicircular canals 118, and auditory nerve 120. “Cochlea” means “snail” in Latin; the cochlea gets its name from its distinctive coiled up shape. The pharyngotympanic tube 122 is in fluid communication with the eustachian tube and helps to control pressure within the middle ear generally making it equal with ambient air pressure.

Sound waves enter the ear canal 112 and make the tympanic membrane 114 vibrate. This action moves the tiny chain of auditory bones 116 (ossicles—malleus, incus, stapes) in the middle ear 104. The last bone in this chain contacts the membrane window of the cochlea 108 and makes the fluid in the cochlea 108 move. The fluid movement then triggers a response in the auditory nerve 120.

Hearing assistance devices, such as hearing aids and hearables (e.g., wearable earphones), can include an enclosure, such as a housing or shell, within which internal components are disposed. Components of a hearing assistance device herein can include a control circuit, digital signal processor (DSP), memory (such as non-volatile memory), power management circuitry, a data communications bus, one or more communication devices (e.g., a radio, a near-field magnetic induction device), one or more antennas, one or more microphones, a receiver/speaker, and various sensors as described in greater detail below. More advanced hearing assistance devices can incorporate a long-range communication device, such as a BLUETOOTH® transceiver or other type of radio frequency (RF) transceiver.

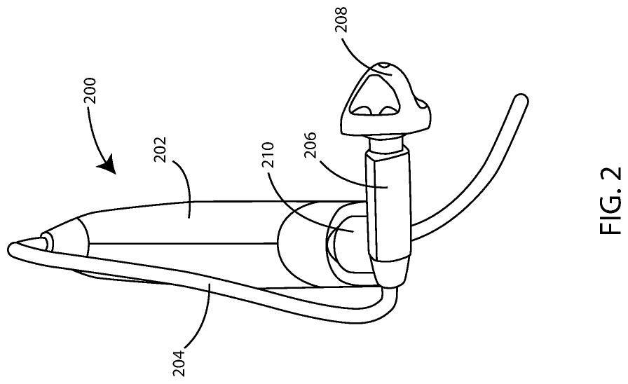

Referring now to FIG. 2, a schematic view of a hearing assistance device 200 is shown in accordance with various embodiments herein. The hearing assistance device 200 can include a hearing device housing 202. The hearing device housing 202 can define a battery compartment 210 into which a battery can be disposed to provide power to the device. The hearing assistance device 200 can also include a receiver 206 adjacent to an earbud 208. The receiver 206 an include a component that converts electrical impulses into sound, such as an electroacoustic transducer, speaker, or loud speaker. A cable 204 or connecting wire can include one or more electrical conductors and provide electrical communication between components inside of the hearing device housing 202 and components inside of the receiver 206.

The hearing assistance device 200 shown in FIG. 2 is a receiver-in-canal type device and thus the receiver is designed to be placed within the ear canal. However, it will be appreciated that may different form factors for hearing assistance devices are contemplated herein. As such, hearing assistance devices herein can include, but are not limited to, behind-the-ear (BTE), in-the ear (ITE), in-the-canal (ITC), invisible-in-canal (IIC), receiver-in-canal (RIC), receiver in-the-ear (RITE) and completely-in-the-canal (CIC) type hearing assistance devices.

Hearing assistance devices of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4. 2 or 5.0) specification, for example. It is understood that hearing assistance devices of the present disclosure can employ other radios, such as a 900 MHz radio. Hearing assistance devices of the present disclosure can be configured to receive streaming audio (e.g., digital audio data or files) from an electronic or digital source. Representative electronic/digital sources (also referred to herein as accessory devices) include an assistive listening system, a TV streamer, a radio, a smartphone, a cell phone/entertainment device (CPED) or other electronic device that serves as a source of digital audio data or files.

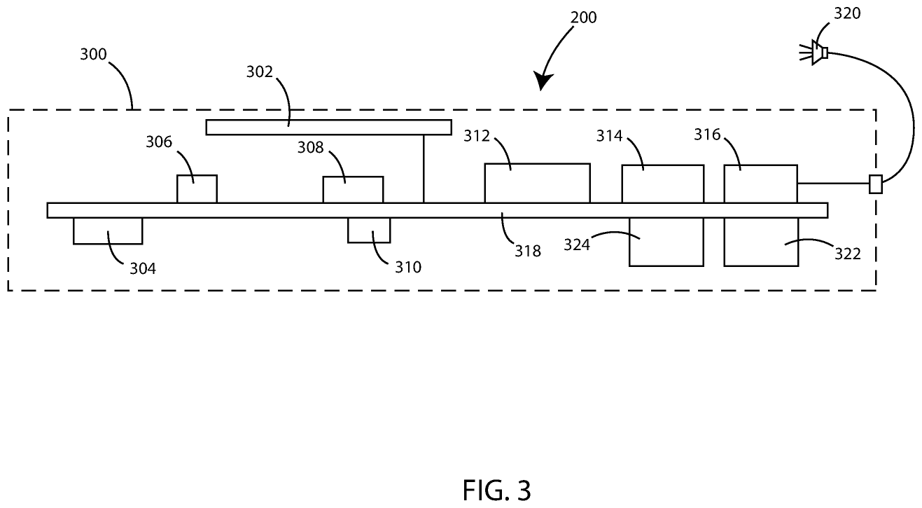

Referring now to FIG. 3, a schematic block diagram is shown with various components of a hearing assistance device in accordance with various embodiments. The block diagram of FIG. 3 represents a generic hearing assistance device for purposes of illustration. The hearing assistance device 200 shown in FIG. 3 includes several components electrically connected to a flexible mother circuit 318 (e.g., flexible mother board) which is disposed within housing 300. A power supply circuit 304 can include a battery and can be electrically connected to the flexible mother circuit 318 and provides power to the various components of the hearing assistance device 200. One or more microphones 306 are electrically connected to the flexible mother circuit 318, which provides electrical communication between the microphones 306 and a digital signal processor (DSP) 312. Among other components, the DSP 312 incorporates or is coupled to audio signal processing circuitry configured to implement various functions described herein. A sensor package 314 can be coupled to the DSP 312 via the flexible mother circuit 318. The sensor package 314 can include one or more different specific types of sensors such as those described in greater detail below. One or more user switches 310 (e.g., on/off, volume, mic directional settings) are electrically coupled to the DSP 312 via the flexible mother circuit 318.

An audio output device 316 is electrically connected to the DSP 312 via the flexible mother circuit 318. In some embodiments, the audio output device 316 comprises a speaker (coupled to an amplifier). In other embodiments, the audio output device 316 comprises an amplifier coupled to an external receiver 320 adapted for positioning within an ear of a wearer. The external receiver 320 can include an electroacoustic transducer, speaker, or loud speaker. The hearing assistance device 200 may incorporate a communication device 308 coupled to the flexible mother circuit 318 and to an antenna 302 directly or indirectly via the flexible mother circuit 318. The communication device 308 can be a BLUETOOTH® transceiver, such as a BLE (BLUETOOTH® low energy) transceiver or other transceiver (e.g., an IEEE 802.11 compliant device). The communication device 308 can be configured to communicate with one or more external devices, such as those discussed previously, in accordance with various embodiments. In various embodiments, the communication device 308 can be configured to communicate with an external visual display device such as a smart phone, a video display screen, a tablet, a computer, or the like.

In various embodiments, the hearing assistance device 200 can also include a control circuit 322 and a memory storage device 324. The control circuit 322 can be in electrical communication with other components of the device. The control circuit 322 can execute various operations, such as those described herein. The control circuit 322 can include various components including, but not limited to, a microprocessor, a microcontroller, an FPGA (field-programmable gate array) processing device, an ASIC (application specific integrated circuit), or the like. The memory storage device 324 can include both volatile and non-volatile memory. The memory storage device 324 can include ROM, RAM, flash memory, EEPROM, SSD devices, NAND chips, and the like. The memory storage device 324 can be used to store data from sensors as described herein and/or processed data generated using data from sensors as described herein, including, but not limited to, information regarding exercise regimens, performance of the same, visual feedback regarding exercises, and the like.

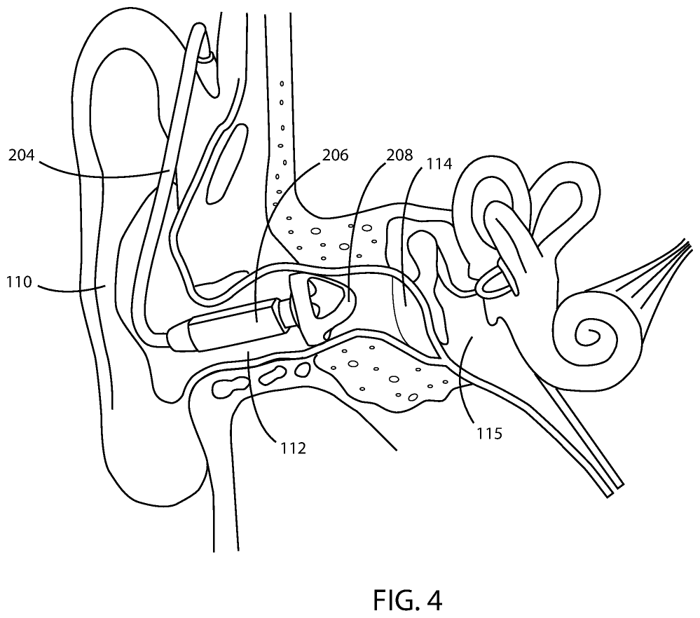

As mentioned regarding FIG. 2, the hearing assistance device 200 shown in FIG. 2 is a receiver-in-canal type device and thus the receiver is designed to be placed within the ear canal. Referring now to FIG. 4, a schematic view is shown of a hearing assistance device disposed within the ear of a subject in accordance with various embodiments herein. In this view, the receiver 206 and the earbud 208 are both within the ear canal 112, but do not directly contact the tympanic membrane 114. The hearing device housing is mostly obscured in this view behind the pinna 110, but it can be seen that the cable 204 passes over the top of the pinna 110 and down to the entrance to the ear canal 112.

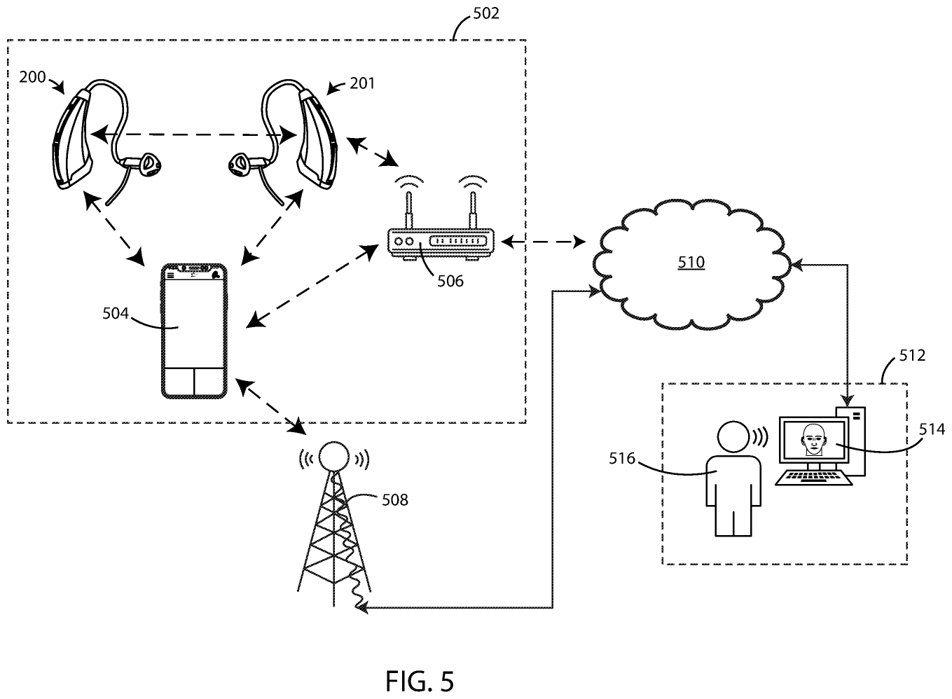

It will be appreciated that data and/or signals can be exchanged between many different components in accordance with embodiments herein. Referring now to FIG. 5, a schematic view is shown of data and/or signal flow as part of a system in accordance with various embodiments herein. In a first location 502, a user (not shown) can have a first hearing assistance device 200 and a second hearing assistance device 201. Each of the hearing assistance devices 200, 201 can include sensor packages as described herein including, for example, an IMU. The hearing assistance devices 200, 201 and sensors therein can be disposed on opposing lateral sides of the subject's head. The hearing assistance devices 200, 201 and sensors therein can be disposed in a fixed position relative to the subject's head. The hearing assistance devices 200, 201 and sensors therein can be disposed within opposing ear canals of the subject. The hearing assistance devices 200, 201 and sensors therein can be disposed on or in opposing ears of the subject. The hearing assistance devices 200, 201 and sensors therein can be spaced apart from one another by a distance of at least 3, 4, 5, 6, 8, 10, 12, 14, or 16 centimeters and less than 40, 30, 28, 26, 24, 22, 20 or 18 centimeters, or by a distance falling within a range between any of the foregoing.

In various embodiments, data and/or signals can be exchanged directly between the first hearing assistance device 200 and the second hearing assistance device 201. An external visual display device 504 with a video display screen, such as a smart phone, can also be disposed within the first location 502. The external visual display device 504 can exchange data and/or signals with one or both of the first hearing assistance device 200 and the second hearing assistance device 201 and/or with an accessory to the hearing assistance devices (e.g., a remote microphone, a remote control, a phone streamer, etc.). The external visual display device 504 can also exchange data across a data network to the cloud 510, such as through a wireless signal connecting with a local gateway device, such as a network router 506 or through a wireless signal connecting with a cell tower 508 or similar communications tower. In some embodiments, the external visual display device can also connect to a data network to provide communication to the cloud 510 through a direct wired connection.

In some embodiments, a care provider 516 (such as an audiologist, physical therapist, a physician or a different type of clinician, specialist, or care provider, or physical trainer) can receive information from devices at the first location 502 remotely at a second location 512 through a data communication network such as that represented by the cloud 510. The care provider 516 can use a computing device 514 to see and interact with the information received. The received information can include, but is not limited to, information regarding the subject's performance of the exercise including, but not limited to, whether or not exercises were performed, accuracy of exercise performance, time spent performing exercises, range of motion, and spatial position information related to IMU and/or accelerometer data, trends related to exercise performance (consistency, accuracy, etc.) and the like. In some embodiments, received information can be provided to the care provider 516 in real time. In some embodiments, received information can be stored and provided to the care provider 516 at a time point after exercises are performed by the subject.

In some embodiments, the care provider 516 (such as an audiologist, physical therapist, a physician or a different type of clinician, specialist, or care provider, or physical trainer) can send information remotely from the second location 512 through a data communication network such as that represented by the cloud 510 to devices at the first location 502. For example, the care provider 516 can enter information into the computing device 514, can use a camera connected to the computing device 514 and/or can speak into the external computing device. The sent information can include, but is not limited to, feedback information, guidance information, future exercise directions/regimens, and the like. In some embodiments, feedback information from the care provider 516 can be provided to the subject in real time. In some embodiments, received information can be stored and provided to the subject at a time point after exercises are performed by the subject or during the next exercise session that the subject performs.

As such, embodiments herein can include operations of sending the feedback data to a remote system user at a remote site, receiving feedback (such as auditory feedback) from the remote system user, and presenting the feedback to the subject. The operation of presenting the auditory feedback to the subject can be performed with the hearing assistance device(s). In various embodiments, the operation of presenting the auditory feedback to the subject can be performed with a hearing assistance device(s) and the auditory feedback can be configured to be presented to the subject as spatially originating (such as with a virtual audio interface described below) from a direction of an end point of the first predetermined movement.

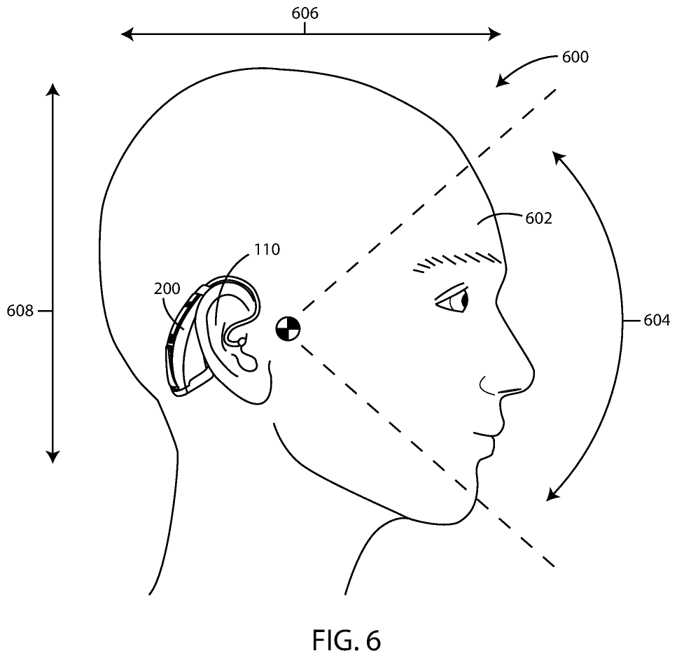

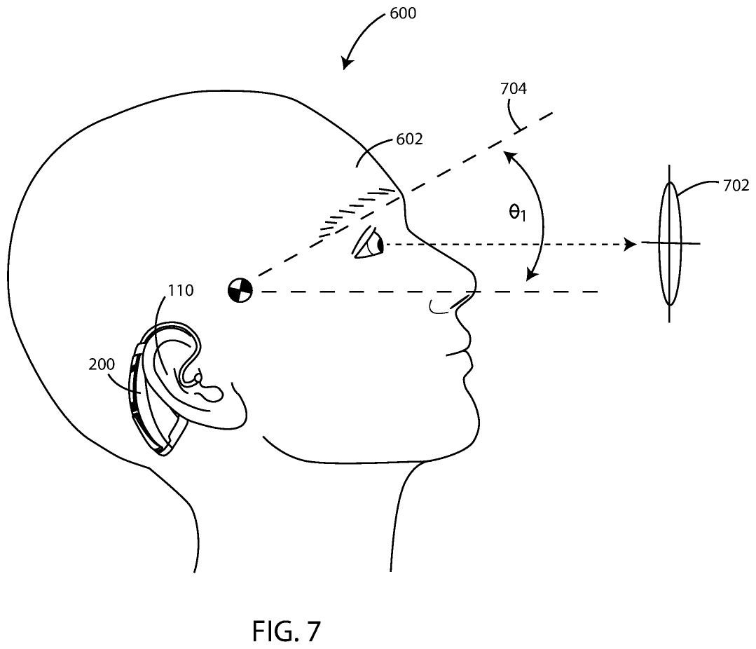

Hearing assistance devices herein can include sensors (such as part of a sensor package 314) to detect movements of the subject wearing the hearing assistance device. Referring now to FIG. 6, a schematic side view is shown of a subject 600 wearing a hearing assistance device 200 in accordance with various embodiments herein. For example, movements detected can include forward/back movements 606, up/down movements 608, and rotational movements 604 in the vertical plane. Such sensors can detect movements of the subject and, in particular, movements of the subject during fixed gaze exercises. Referring now to FIG. 7, a schematic side view of a subject 602 wearing a hearing assistance device 200 and executing a fixed gaze exercise in accordance with various embodiments herein. In this example, the subject 602 is directing their gaze at a fixed target 702 (or fixed spot). As part of a particular movement or segment of a fixed-gaze exercise, the subject 602 has tipped (or rotated) their head backward causing the front of their face to be directed upward along line 704. As such, in this example, the direction of their face and the direction of gaze diverge by angle θ1. Angle θ₁ can vary and can be both positive and negative (e.g., their head can be tipped up or down) at various times during the overall course of the exercise. In some embodiments, angle θ1 can be up to 5, 10, 15, 20, 25, 30, 35, 40, 50, 60, 70, or 80 degrees, or can be an angle that falls within a range wherein any of the foregoing can serve as the upper or lower bound of the range. In some embodiments, a position of maximum movement or rotation can be held for a period of time before the next step of the exercise. In some embodiments, the next step of the exercise can begin immediately after attaining a position of maximum movement or rotation. In some embodiments, a series of movements of the exercise can include a movement of rotating the head so that angle θ1 is positive followed by a movement of rotating the head so that angle θ1 is positive and then repeating this cycle of movements a predetermined number of times.

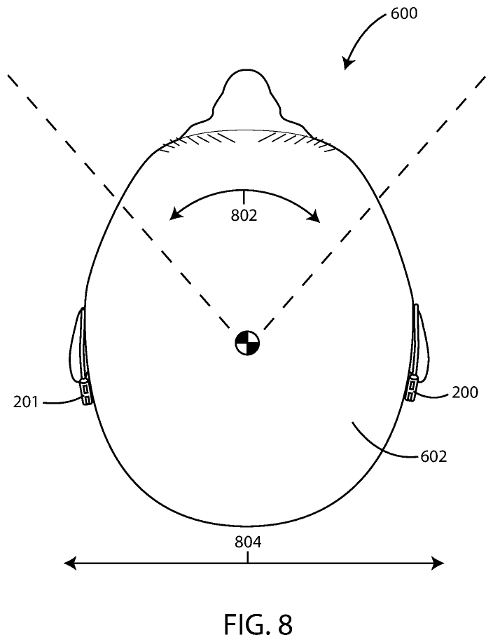

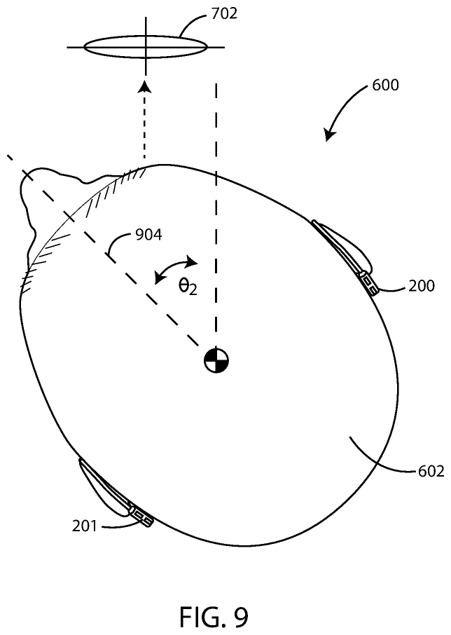

Referring now to FIG. 8, a schematic top view is shown of a subject 600 wearing hearing assistance devices 200, 201 in accordance with various embodiments herein. Movements detected can also include side-to-side movements 804, and rotational movements 802 in the horizontal plane. Referring now to FIG. 9, a schematic top view of a subject 602 wearing hearing assistance devices 200, 201 and executing a fixed gaze exercise in accordance with various embodiments herein. In this example, the subject 602 is directing their gaze at a fixed target 702 (or fixed spot). As part of a particular movement or segment of a fixed-gaze exercise, the subject 602 has rotated their head to their left causing the front of their face to be directed leftward along line 904. As such, in this example, the direction of their face and the direction of gaze diverge by angle θ₂. Angle θ₂ can vary and can be both positive and negative (e.g., their head can be rotated left or right) at various times during the overall course of the exercise. In some embodiments, angle θ₂ can be up to 5, 10, 15, 20, 25, 30, 35, 40, 50, 60, 70, or 80 degrees, or can be an angle that falls within a range wherein any of the foregoing can serve as the upper or lower bound of the range. In some embodiments, a position of maximum movement or rotation can be held for a time period before the next step of the exercise. In some embodiments, the next step of the exercise can begin immediately after attaining a position of maximum movement or rotation. In some embodiments, a series of movements of the exercise can include a movement of rotating the head so that angle θ₂ is positive followed by a movement of rotating the head so that angle θ₂ is positive and then repeating this cycle of movements a predetermined number of times. In some embodiments, the exercise can include moving (rotating or tipping) the subject's head such that both angle θ₁ of FIG. 7 and angle θ₂ of FIG. 9 change at the same time.

In some embodiments, the system and/or devices thereof can evaluate data from sensors and/or a camera in order to detect irregular eye movements when the exercise includes rotation of the front of the head farther than a threshold amount away from the fixed point of eye gaze (e.g., when angle θ₂ is greater than 20, 25, 30, 35, 40, 45, or 50 degrees or equal to a maximum value for the particular subject, or when θ₂ is less than −20, −25, −30, −35, −40, −45, or −50 degrees or equal to a minimum value for the particular subject. In some embodiments, the system and/or devices thereof can evaluate data from sensors and/or a camera to detect rapid eye movements when the exercise includes rotation of the front of the head farther than a threshold amount away from the fixed point of eye gaze. In some embodiments, the system and/or devices thereof can evaluate data from sensors and/or a camera to detect nystagmus when the exercise includes rotation of the front of the head farther than a threshold amount away from the fixed point of eye gaze. In some embodiments, the system and/or devices thereof can evaluate data from sensors and/or a camera to detect, for example, horizontal gaze nystagmus when the exercise includes rotation of the front of the head farther than a threshold amount away from the fixed point of eye gaze. In some embodiments, the system and/or devices thereof can evaluate data from sensors and/or a camera to track movement of both eyes when the exercise includes rotation of the front of the head while maintaining a fixed gaze and comparing movement of the eyes against one another. In some embodiments, the system and/or devices thereof can track smoothness of movement of at least one eye when the exercise includes rotation of the front of the head while maintaining a fixed gaze.

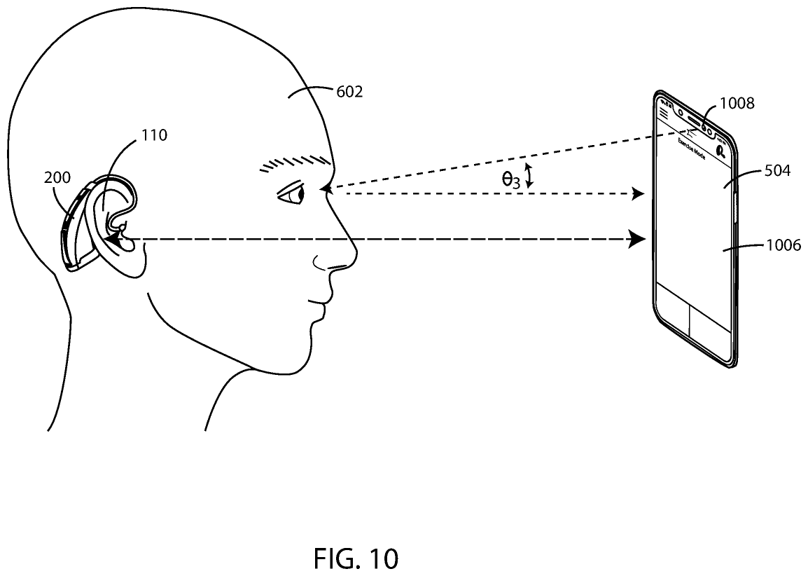

In accordance with various embodiments herein, the hearing assistance device and/or the system can prompt the subject to execute an exercise. The exercise can include one or more predetermined movements while maintaining a fixed point of eye gaze. The hearing assistance device and/or system can track the point of gaze of the subject's eyes using one or more of a camera, an EOG (electrooculogram) sensor, or other device. The hearing assistance device and/or system can generate data representing a measured deviation between the fixed point of eye gaze and the actual tracked point of gaze (in terms of degrees of angular deviation-vertical and/or horizontal, distance of deviation, torsion, or the like). Measured deviations can be used for various purposes including, but not limited to, scoring accuracy of movements/exercises, providing feedback to the subject, providing feedback to a care provider or exercise leader, trending the subject's condition over time, scoring points in a game, providing control inputs to a game, impacting or setting frequencies/schedules of exercise repetitions, and the like. Referring now to FIG. 10, a schematic view is shown of a subject 602 wearing a hearing assistance device 200 and receiving visual feedback from an external visual display device 504 in accordance with various embodiments herein. The external visual display device 504 can include a display screen 1006 and one or more cameras 1008. In some embodiments, the display screen 1006 can be a touch screen. The display screen 1006 can display various pieces of information to the subject 602 including, but not limited to, instructions for exercises, visual feedback regarding the fidelity with which the subject 602 is performing the exercises, a target or icon for the subject to focus their gaze on, information regarding the progress of the subject 602 through a particular set of exercises, the remaining time to complete a particular set of exercises, current feedback from a care provider (remote or local), or the like.

A first camera 1008 can be positioned to face away from the display screen 1006 and back toward the subject 602 (in some embodiments, the camera could also be facing the display, with the subject between the camera and the display screen—using the display itself as a spatial reference or the camera could be on the back of the display and track movement of the display relative to visual objects in the environment). The camera 1008 can be used to capture an image or images of the subject's 602 face and, in some cases, the subject's 602 eyes. In some embodiments, the camera 1008 can be used to capture image(s) including the positioning of subject's 602 face, pupil, iris, and/or sclera. Such information can be used to calculate the direction of the subject's 602 face and/or gaze. In some embodiments, such information can also be used to calculate angle, speed and direction of nystagmus. Aspects of nystagmus detection and characterization are described in commonly-owned U.S. Publ. Pat. Appl. No. 2018/0228404, the content of which is herein incorporated by reference. In some embodiments, such information can specifically be used to calculate the direction of the subject's 602 face and/or gaze with respect to the camera 1008. Aspects regarding such calculations are described in U.S. Publ. Appl. Nos. 2012/0219180 and 2014/0002586; the content of which is herein incorporated by reference. In some embodiments, information from the camera can be used to calculate the angle, speed, and direction of nystagmus. In some embodiments, information from other sensors (such as an EOG sensor) can be used in combination with data from the camera to more accurately calculate the direction of the subject's face, gaze, or another aspect described herein.

While not intending to be bound by theory, it is believed that the accuracy of gaze determination can be enhanced if the camera 1008 is positioned so as to minimize an angle (θ₃) in the vertical plane formed between a first line connecting the camera 1008 and the subject's pupils and a second line connecting the display screen 1006 (or a specific point thereon such as a midpoint or a point of visual focus) and the subject's pupils. In some embodiments, the camera 1008 is positioned such that the described angle is less than 20, 15, 10, 8, 6, 5, 4, 3, 2, or 1 degrees, or an amount falling within a range between any of the foregoing. In some embodiments, camera 1008 is positioned such that the distance between the camera 1008 and the display screen 1006 (or a specific point thereon such as a midpoint or a point of visual focus) is less than 30, 25, 20, 18, 16, 14, 12, 10, 8, 6, 5, 4, 3, 2, or 1 cm, or a distance falling within a range between any of the foregoing.

In various embodiments herein, the system and/or devices can be configured to detect performance of an exercise or movements thereof by evaluating data from at least one of a camera, an IMU, and another type of sensor. In some embodiments, aspects of the subject can be detected to monitor for issues of concern. For example, in some scenarios, the pupils may dilate prior to syncope or another type of loss-of-consciousness event. In some embodiments, if warning signs such as pupil dilation or nystagmus are detected, the system can prompt the subject to cease performing the exercise. In some embodiments, camera data can be evaluated for evidence of pupil dilation or nystagmus using camera data after performance of the exercise is first detected (using accelerometer data, camera data, and/or another type of sensor data) and prompting the subject to cease performing the exercise.

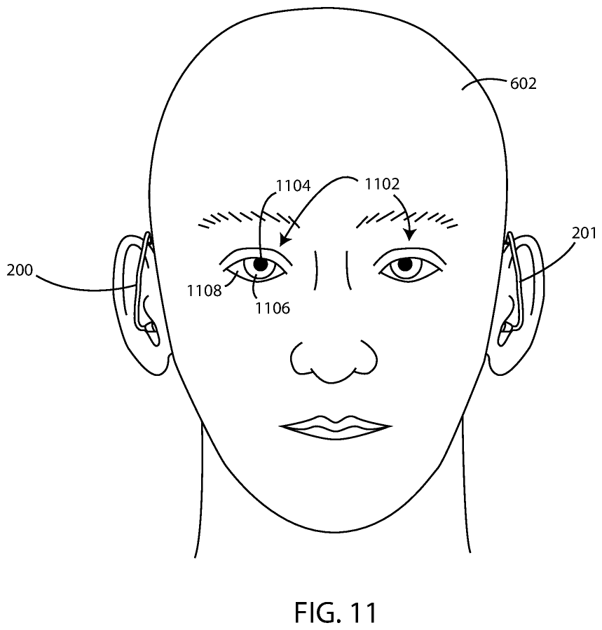

Referring now to FIG. 11, a schematic frontal view is shown of a subject 602 wearing hearing assistance devices 200, 201 in accordance with various embodiments herein. The subject's 602 eyes 1102 include pupils 1104, iris 1106, and sclera 1108 (or white portion). Identifying the position of these and other eye components and facial components can be used to determine the direction of gaze and/or direction the face is pointing as described above. In some embodiments, the size of the pupils 1104 can be monitored using camera data to detect any changes that occur during an exercise.

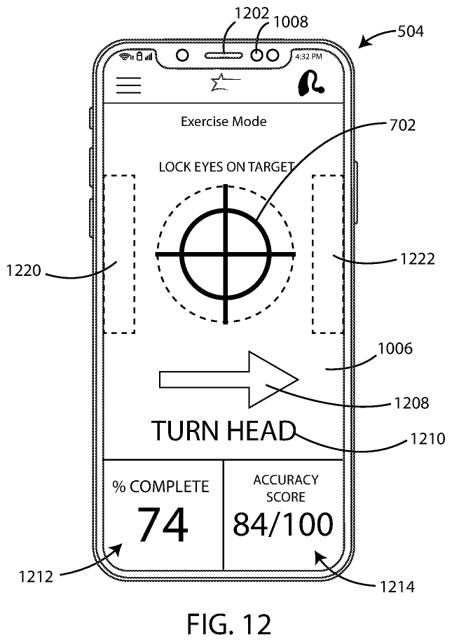

Referring now to FIG. 12, a schematic view is shown of an external visual display device 504 and elements of the display screen 1006 thereof. The external visual display device 504 can include a speaker 1202. The external visual display device 504 can generate and/or display a target image, instructions, and/or a feedback image or elements using camera data to determine the direction of gaze.

The external visual display device 504 can display a target 702 (or focus spot) on the display screen 1006. The target 702 can take on many different specific forms including, but not limited to, a reticle, a shape (polygonal or non-polygonal), a user-selectable graphic object, or the like. In some embodiments, the display device 504 can display graphic elements 1220, 1222 on the display screen 1006. Graphic elements 1220, 1222 can be directionally associated (on the left and right in this view but could also be on the top and/or bottom). In some embodiments, graphic elements 1220, 1222 can be visually altered to signal directional information to the subject 602. For example, if the next movement in the exercise involves rotating the head to the right, then the graphic element 1222 on the right side (as judged from the perspective of the subject) can be flashed, altered in color or brightness, or otherwise visually change to indicate to the subject which way to rotate their head.

In some embodiments, a target 702 can be on a wall or other structure and the target can be monitored with a camera on one side of a device while the camera on the other side of the device can be used to monitor the subject's eyes.

In some embodiments, the external visual display device 504 can display a directional icon 1208, such as an arrow, indicating the direction that the patient should be moving their head. The directional icon can be provided as a mirror image so that the arrow can be directly followed in order to result in the proper movement of the patient's head (e.g., if the patient currently needs to rotate their head to the right in order to follow the determined movement of the exercise the arrow on the external visual display device 504 can be pointing to the left side of the screen as judged from the perspective of the external visual display device facing back toward the subject).

In various embodiments, the external visual display device 504 can display a textual instruction 1210 guiding the subject to perform the determined movement of the exercise, such as “Turn Head” or “Turn Head 90° Right”.

In some embodiment, the external visual display device 504 can display one or more written words with the object being for the user to be able to read the words despite movement (such as head movement and/or display screen movement). In this context, a goal for the user would be to increase the speed by which the user can move the display and/or their head while still being able to read the words. In some embodiments, the system can present a word and then monitor for a verbal response from the user (such as the user saying the word aloud), identify what word the user has said, and then score for accuracy against the word that was displayed and thereby determine if the user is able to read the text at a given speed of focal point movement (whether due to head movement or display screen movement). Identifying spoken words can be performed in various ways. In some embodiments, a speech recognition API can be utilized to identify spoken words. In some embodiments, a Hidden Markov Model can be used to identify spoken words. In some embodiments, a dynamic time warping approach can be used to identify spoken words. In some embodiments, a neural network can be used to identify spoken words. The speed of head movement during the exercise can be measured in various ways, such as using a motion sensor, IMU, or accelerometer as described herein. If a threshold amount of accuracy is achieved for a given speed or range of speeds, the system can prompt the user to try to increase speed. Conversely, if a threshold amount of accuracy is not achieved for a given speed or range of speeds, the system can prompt the user to try to slow down.

Various other pieces of data regarding the exercise or movements thereof can be displayed on the external visual display device 504 and/or auditorily via the hearing assistance device 200. For example, information regarding the state of completion 1212 of the exercise can be displayed on the external visual display device 504. Such state of completion 1212 information can be displayed in the form of a current percent of completion of the exercise session, an elapsed time of the exercise session so far, a remaining time of the exercise session, or the like.

Information regarding the accuracy of the patient's performance of the exercise 1214 can also be displayed on the external visual display device 504. In some embodiments, the accuracy of the patient's performance of the exercise 1214 can be displayed and reflected as a calculated score. Many different techniques for calculating a score can be used. By way of example, in the context of a fixed-gaze exercise, the score can be calculated based on deviation of their gaze from the fixed point of focus during the exercise. If the gaze of the subject deviates by less than a threshold amount, such as less than 5%, then they may earn the full number of possible points for the movement. If the exercise contains ten distinct movements (as merely one example) and the total number of possible points is 100, then executing 9/10 of the movements with a deviation of less than 5% can result in a score of 90/100. As another example, the average deviation for all movements in the exercise can be used to calculate a score. For example, if the average deviation during all movements in the exercise is 5%, then a score can be determined as 95/100. Many different scoring approaches can be used with embodiments herein. The score of the patient's performance of the exercise 1214 shown on the external visual display device 504 can reflect an average of accuracy scores for each movement performed so far during the current exercise session. In various embodiments, the accuracy of the patient's performance of the exercise 1214 shown on the external visual display device 504 can change visually based on the current degree of accuracy. For example, current scores or average scores above 90 can be shown in blue or green and scores below 50 can be shown in red. Many visual display options are contemplated herein.

In various embodiments herein, the system and/or devices can be configured to calculate a trend using measured deviations or scores, such as those based on measured deviations or other determinants of exercise performance accuracy). For example, the system can calculate an average deviation or other determinant of exercise performance accuracy for the most recently completed exercise session and compare it with deviations, scores or results from previous days. In some embodiments, the information for the most recently completed exercise can be compared with a moving average or product of statistical calculation (such as a standard deviation) based on deviations, scores, or results from previous days or other statistics relative to previous performance. In some embodiments, the trend can be reported to a remote care provider or leader. In some embodiments, a warning notification can be issued and/or sent to a remote care provider, leader or designated emergency contact if the trend indicates a worsening or decline of the subject's condition that exceeds a threshold value. In some embodiments, a worsening of exercise performance accuracy that crosses threshold in terms of magnitude and/or length of time (e.g., over what time period the supra-threshold performance lasts) can be interpreted by the system and/or devices as a marker of a vestibular decompensation event or process. In some embodiments, the system and/or devices can also consider physiological markers, eye movement, health sensor data, and the like when determining whether a decompensation event or process in occurring.

In some embodiments, incentives can be awarded to the subject based on their performance of exercises and/or accuracy of their performance. In some embodiments herein, the system and/or devices can be configured to detect performance of the exercise by evaluating data from at least one of a camera and an IMU and award an electronic incentive to the subject if a threshold of exercise performance is met. The incentives can be real or virtual (electronic points, currency, etc.). In some embodiments, the system and/or devices herein can be configured to award an electronic incentive to the subject if the measured deviation between the fixed point of eye gaze and the tracked point of gaze crosses a threshold amount.

In some embodiments, the exercise can be turned into a game wherein control elements/inputs for the game can be linked to sensed movements/actions of the subject while performing exercises including, but not limited to, movement or rotation of the head, directional gaze of the eyes, etc. Control elements can include, but are not limited to, virtual button presses/inputs, directional inputs, and the like. For example, in various embodiments herein, a target-type game (e.g., throwing a dart at a board, shooting an arrow at a target, etc.) can be played wherein elements of a fixed-gaze exercise are used as game input controls. In a particular example, if the exercise involves rotating the head while maintaining the fixed gaze, then throwing or otherwise shooting the object in the game can be triggered when the system or device senses that the subject has rotated or tipped their head by at least a predetermined amount in the direction required by the particular movement and the point on a target board in the game where the object lands is based on the direction of the subject's gaze when the throwing or otherwise shooting the object was triggered.

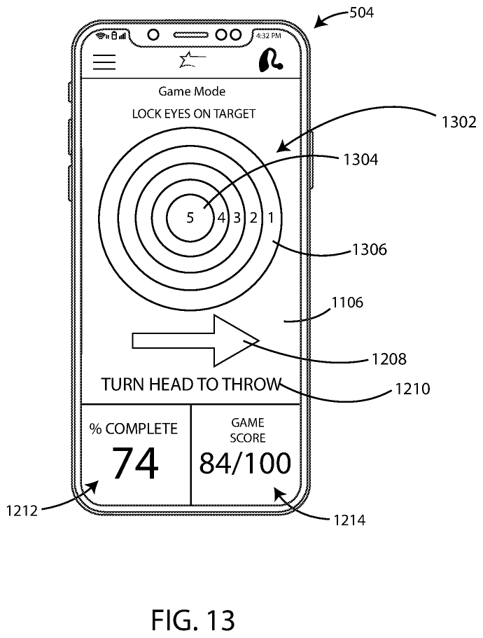

Referring now to FIG. 13, a schematic view is shown of an external visual display device 504 and elements of the display screen 1006 thereof in accordance with various embodiments herein. The external visual display device 504 can generate and/or display a target image, instructions, and/or a feedback image or elements using camera data to determine the direction of gaze and/or detect nystagmus. A target image 1302 can be displayed. The target image 1302 can include areas corresponding to more points 1304 near the center thereof and areas corresponding to fewer points 1306 farther away from the center of the target image 1302. In some embodiments, data from various sensors described herein (including but not limited to IMUs or accelerometers) can be used to detect rotation or movement of the subject's head associated with a particular movement of an exercise. Simultaneously, the direction of the subject's gaze can be tracked as described elsewhere herein. When the system or device senses that the subject has rotated or tipped their head by at least a predetermined threshold amount consistent with the particular movement the subject is to be performing, then the current direction of the subject's gaze can be matched against a specific point on the target board and various actions can be taken such as assigning points to the user and/or visually superimposing a mark or object over the spot on the target image 1302 that matches where the subject's gaze was directed when their movement or rotation triggered an action in the game. Many different game play options are contemplated herein including triggering game control actions by discrete elements of performance of an exercise.

In various embodiments herein, systems for remote care providers or exercise leaders to provide direction or guidance to a plurality of subjects are included. In some embodiments, a remote care provider can provide prompts from their remote location to subjects to execute an exercise or a movement thereof. Such prompts can be provided in real time or can be delayed such that the prompt is initiated at a first time and is delivered to the subject(s) at a second time that is later than the first time by an amount time that is minutes, hours or days. In some embodiments, a leader or care provider in a remote location simultaneously prompts a plurality of subjects to execute the exercise.

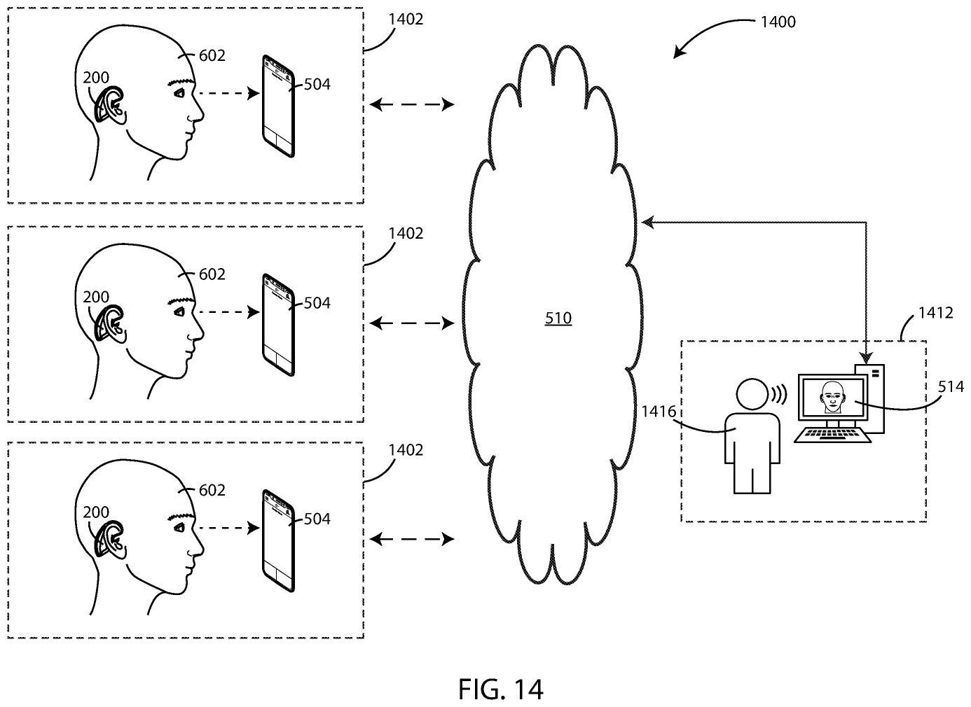

Referring now to FIG. 14, a schematic view is shown of a system 1400 in accordance with various embodiments herein. The system 1400 can include a leader (or care provider—such as an audiologist, physical therapist, a physician or a different type of clinician, specialist, or care provider, or physical trainer) 1416 at a remote location 1412. The leader 1416 can use a computing device 514 (or other device capable of receiving input) to input information including prompts, directions, and/or guidance regarding exercises or discrete movements thereof. These inputs can be processed and then conveyed (in various forms) through a data communication network such as that represented by the cloud 510. The prompts, directions, and/or guidance can then be conveyed to a plurality of locations 1402 wherein subjects 602 are located. The subjects 602 can receive the information from the leader 1416 via hearing assistance devices 200 and/or external visual display devices 504. In some embodiments, the leader 1416 can use the computing device 514 to see and interact with the subjects 602. Information from the locations 1402 can be transmitted to the leader including, but not limited to, information regarding the subjects' performance of the exercises including, but not limited to, whether or not exercises were performed, accuracy of exercise performance, time spent performing exercises, range of motion, spatial position information related to IMU and/or accelerometer data, trends related to exercise performance (consistency, accuracy, etc.) and the like.

### Methods

Various methods are included herein. In some embodiments, methods of providing vestibular therapy and/or exercises to a subject are included herein. In some embodiments, method steps described can be executed as a series of operations by devices described herein.

In an embodiment, a method of providing vestibular therapy to a subject is included. The method can include prompting the subject to execute an exercise, the exercise comprising a predetermined movement while maintaining a fixed point of eye gaze. The method can further include tracking the point of gaze of the subject's eyes using a camera. The method can further include generating data representing a measured deviation between the targeted fixed point of eye gaze and the actual tracked point of gaze. In some embodiments, the predetermined movement can include including movement and/or rotation of the head.

In some embodiments, methods herein can include providing feedback to the subject based on the measured deviation. In some embodiments, methods herein can include generating a score based on (and/or statistics related to) a measured deviation between the targeted fixed point of eye gaze and the actual tracked point of gaze. In some embodiments, the method can include sending information regarding the measured deviation to a remote system user such as a care provider.

In some embodiments, the methods can include storing the measured deviation and comparing it with measured deviations from exercises performed on previous days. In some embodiments, methods herein can include calculating a trend using the measured deviation and previously measured deviations from exercises performed on previous days. In some embodiments, methods herein can include reporting the trend to a remote care provider. In some embodiments, methods herein can include issuing a warning notification if the trend indicates a worsening of the subject's condition.

In some embodiments, methods herein can include setting a frequency for repeating the exercise based in part on a measured deviation between the targeted fixed point of eye gaze and the actual tracked point of gaze.

In some embodiments, methods herein can include tracking movement of the subject using an IMU disposed in a fixed position relative to their head. In some embodiments, methods herein can include tracking movement of the subject using a camera. In some embodiments, methods herein can include providing visual feedback to the subject through an external video output device reflecting a measured deviation between the targeted fixed point of eye gaze and the actual tracked point of gaze. In some embodiments, methods herein can include providing visual feedback to the subject through an external video output device if the measured deviation exceeds a threshold value. In some embodiments, methods herein can include auditory guidance to the subject during the exercise. In some embodiments, methods herein can include detecting performance of the exercise by evaluating data from at least one of a camera and an IMU. In some embodiments, methods herein can include evaluating external camera data for evidence of pupil dilation after performance of the exercise is first detected and prompting the subject to cease performing the exercise.

In some embodiments, methods herein can include detecting irregular eye movements when the exercise includes rotation of the front of the head farther than a threshold amount away from the fixed point of eye gaze. In some embodiments, methods herein can include detecting rapid eye movements and/or nystagmus when the exercise includes rotation of the front of the head farther than a threshold amount away from the fixed point of eye gaze. In some embodiments, methods herein can include detecting nystagmus when the exercise includes rotation of the front of the head farther than a threshold amount away from the fixed point of eye gaze. In some embodiments, methods herein can include detecting horizontal gaze nystagmus when the exercise includes rotation of the front of the head farther than a threshold amount away from the fixed point of eye gaze.

In some embodiments, methods herein can include tracking movement of both eyes when the exercise includes rotation of the front of the head while maintaining a fixed gaze and comparing movement of the eyes against one another. In some embodiments, methods herein can include tracking smoothness of movement of at least one eye when the exercise includes rotation of the front of the head while maintaining a fixed gaze.

In some embodiments, methods herein can include prompting the subject to execute an exercise according to a predetermined schedule input by a care provider. In some embodiments, methods herein can include changing the predetermined schedule based on at least one of the accuracy of exercise performance, the frequency of exercise performance, changes in health status, other metrics of previous exercise sessions, or the like. In some embodiments, methods herein can include sending information regarding schedule changes and/or at least one of the accuracy of exercise performance and the frequency of exercise performance back to the care provider.

In some embodiments, methods herein can include queuing prompts according to a predetermined schedule and triggering the prompt after detecting sedentary behavior of the subject. In some embodiments, methods herein can include prompting the subject is performed by queuing the prompt according to a predetermined schedule and triggering the prompt after detecting sedentary behavior of the subject if the sedentary behavior is detected during a predefined time window. In some embodiments, methods herein can include prompting the subject and/or remote care providers if nystagmus is detected in the subject.

In some embodiments, methods herein can include prompting the subject to execute an exercise comprises receiving a prompt from a remote location. In some embodiments, methods herein can include prompting the subject to execute an exercise comprises receiving a prompt from a leader in a remote location, which can be in real time or non-real time. In some embodiments, methods herein can include further include detecting performance of the exercise by evaluating data from at least one of a camera and an IMU and awarding an electronic incentive to the subject if a threshold of exercise performance is met.

### Prompting and Timed or Periodic Initiation of Exercises

In accordance with various embodiments herein, the system and/or devices thereof can prompt the subject to execute exercises. In one scenario, a care provider may set a schedule (provided as input) for performing exercises (such as three times every day) and this schedule can be stored within the system and/or devices thereof. The device can then prompt the subject to perform the exercises consistent with the predetermined schedule. In some embodiments, the system and/or devices thereof may store information regarding a normal awake period (e.g., hours when the subject is normally awake during a 24-hour cycle) and then distribute prompts throughout the awake period. The awake period can be provided as input to the device and/or system and can be stored in the memory thereof. However, in some embodiments, the system and/or devices can calculate the normal awake period for the subject by evaluating data from sensors described herein, including, but not limited to, accelerometer data. After calculating normal awake periods, the system and/or devices thereof can then distribute prompts throughout the awake period.

In some embodiments, the predetermined schedule can be changed by the system (increase frequency, decrease frequency, omit an exercise session, add an exercise session, etc.). For example, in some embodiments, the system and/or devices thereof can be configured to change the predetermined schedule based on at least one of the accuracy of exercise performance, the frequency of exercise performance, or other metrics, such as health-related metrics, or other markers that could indicate improvement or worsening of a condition or status. In some embodiments, the system and/or device thereof can change the predetermined schedule if an occurrence of nystagmus is detected by the system and/or devices.

In some embodiments, prompts can be queued according to a schedule but not actually delivered to the subject (via a visual and/or an auditory notification) until one or more specific events are detected or a particular absence of one or more events is detected. By way of example, in some embodiments, the system and/or devices thereof can first queue the prompt according to a predetermined schedule and then trigger delivery of the prompt after detecting sedentary behavior of the subject. In some embodiments, the system and/or devices thereof can first queue the prompt according to a predetermined schedule and then trigger delivery of the prompt after detecting sedentary behavior of the subject, if the sedentary behavior is detected during a predefined time window, such as a normal awake period. Sedentary behavior can be detected in various ways including, but not limited to, accelerometer data that crosses a threshold value, heart rate data that crosses a threshold value, blood pressure data that crosses a threshold value, or the like. In some embodiments, prompting the subject can be performed if nystagmus is detected in the subject.

### Sensors

According to various embodiments, hearing assistance devices herein can include a sensor package or arrangement configured to sense various aspects such as the movement of the wearer during each of the body actions required to implement a predetermined corrective or therapeutic maneuver, physical therapy or exercise routine. The sensor package can comprise one or a multiplicity of sensors, such one or more of an inertial measurement unit (IMU), accelerometer, gyroscope, barometer, magnetometer, microphone, optical sensor, camera, electroencephalography (EEG) and eye movement sensor (e.g., electrooculogram (EOG) sensor). In some embodiments, the sensor package can comprise one or more additional sensors that are external of the hearing assistance device. The one or more additional sensors can comprise one or more of an IMU, accelerometer, gyroscope, barometer, magnetometer, an acoustic sensor, eye motion tracker, EEG or myographic potential electrode (e.g., EMG), heart rate monitor, and pulse oximeter. For example, the one or more additional sensors can include a wrist-worn or ankle-worn sensor package, or a sensor package supported by a chest strap. In some embodiments, the additional sensor can include a camera, such as one embedded within a device such as glasses frames.

The sensor package of a hearing assistance device is configured to sense movement of the wearer as he or she executes each action of a predetermined corrective or therapeutic maneuver, physical therapy or exercise routine. Data produced by the sensor package is operated on by a processor of the hearing assistance device to determine if a specified action was successfully or unsuccessfully executed by the wearer.

According to various embodiments, the sensor package can include one or more of an IMU, and accelerometer (3, 6, or 9 axis), a gyroscope, a barometer, a magnetometer, an eye movement sensor, a pressure sensor, an acoustic sensor, a heart rate sensor, an electrical signal sensor (such as an EEG, EMG or ECG sensor), a temperature sensor, a blood pressure sensor, an oxygen saturation sensor, an optical sensor, and the like. As used herein the term “inertial measurement unit” or “IMU” shall refer to an electronic device that can generate signals related to a body's specific force and/or angular rate. IMUs herein can include one or more of an accelerometer (3, 6, or 9 axis) to detect linear acceleration and a gyroscope to detect rotational rate. In some embodiments, an IMU can also include a magnetometer to detect a magnetic field. The eye movement sensor may be, for example, an electrooculographic (EOG) sensor, such as an EOG sensor disclosed in commonly owned U.S. Pat. No. 9,167,356, which is incorporated herein by reference. The pressure sensor can be, for example, a MEMS-based pressure sensor, a piezo-resistive pressure sensor, a flexion sensor, a strain sensor, a diaphragm-type sensor and the like. The temperature sensor can be, for example, a thermistor (thermally sensitive resistor), a resistance temperature detector, a thermocouple, a semiconductor-based sensor, an infrared sensor, or the like. The blood pressure sensor can be, for example, a pressure sensor. The heart rate sensor can be, for example, an electrical signal sensor, an acoustic sensor, a pressure sensor, an infrared sensor, an optical sensor, or the like. The oxygen saturation sensor can be, for example, an optical sensor, an infrared sensor, or the like. The electrical signal sensor can include two or more electrodes and can include circuitry to sense and record electrical signals including sensed electrical potentials and the magnitude thereof (according to Ohm's law where V=IR) as well as measure impedance from an applied electrical potential.

The sensor package can include one or more sensors that are external to the hearing assistance device. In addition to the external sensors discussed hereinabove, the sensor package can comprise a network of body sensors (such as those listed above) that sense movement of a multiplicity of body parts (e.g., arms, legs, torso).

### Virtual Audio Interfaces

In some embodiments, a virtual audio interface can be used to provide auditory feedback to a subject in addition to visual feedback as described elsewhere herein. The virtual audio interface can be configured to synthesize three-dimensional (3-D) audio that guides the wearer in performing specific physical movements of a predetermined corrective or therapeutic maneuver, physical therapy or exercise routine.

According to some embodiments, the virtual audio interface can generate audio cues comprising spatialized 3-D virtual sound emanating from virtual spatial locations that serve as targets for guiding wearer movement. The wearer can execute a series of body movements in a direction and/or extent indicated by a sequence of virtual sound 5 targets. The sound generated at the virtual spatial locations can be any broadband sound, such as complex tones, noise bursts, human speech, music, etc. or a combination of these and other types of sound. In various embodiments, the virtual audio interface is configured to generate binaural or monaural sounds, alone or in combination with spatialized 3-D virtual sounds. The binaural and monaural sounds can be any of those listed above including single-frequency tones.

In other embodiments, the virtual audio interface is configured to generate human speech that guides the wearer in performing specific physical movements of a predetermined corrective or therapeutic maneuver, physical therapy or exercise routine. The speech can be synthesized speech or a pre-recording of real speech. In embodiments that employ a single hearing assistance device (for one ear), for example, the virtual audio interface generates monaural sound in the form of speech, which can be accompanied by other sounds, such as single or multi-frequency tones, noise bursts or music. In embodiments that employ two hearing assistance devices (one device for each ear), the virtual audio interface can generate monaural or binaural sound in the form of speech, which can be accompanied by other sounds, such as single or multi-frequency tones, noise bursts or music. The virtual audio interface can display (play back) spoken instructions to guide the wearer though specific physical movements of a predetermined corrective or therapeutic maneuver, physical therapy or exercise routine. Further aspects of virtual audio interfaces are described in commonly owned U.S. patent application Ser. No. 15/589,298, titled “Hearing Assistance Device Incorporating Virtual Audio Interface for Therapy Guidance”, the content of which is herein incorporated by reference in its entirety.

### Exercise Movements

In accordance with various embodiments herein, hearing assistance devices can be configured to guide the wearer of a hearing assistance device through a prescribed series of body movements or actions in accordance with a predetermined corrective or therapeutic maneuver, physical therapy or exercise routine. A maneuver, physical therapy or exercise routine involves a prescribed series of body movements or actions that can be implemented by the wearer of a hearing assistance device in an attempt to correct or treat a physiologic disorder or execute a physical fitness routine. Exercises (or routines or maneuvers herein) can include, but are not limited to, habituation exercises, gaze stabilization exercises, and balance training exercises. In some embodiments, the exercises are specifically fixed gaze exercises. Exercises can include a series of actions including one or more of turning their head in a specified direction by a specified amount, moving their head in a specific direction by a specified amount, assuming different postures, etc. In various embodiments, any of these actions can be performed by the subject while they attempt to fix their gaze on a stationary point or object. Gaze stabilization exercises can be used to improve control of eye movements, so vision can be clear during head movement. These exercises are appropriate for patients who report problems seeing clearly because their visual world appears to bounce or jump around, such as when reading or when trying to identify objects in the environment, especially when moving about.

Guidance and/or feedback herein can include auditory guidance, visual guidance, or auditory and visual guidance. Audio guidance can include any one or a combination of different sounds, such as tones, noise bursts, human speech, animal/natural sounds, synthesized sounds, and music, among other sounds.

For example, the virtual audio interface can display spoken words that instruct the wearer to assume a specific position, such as lying down, standing or sitting up. A spoken instruction can be displayed that requests the wearer to move a specific body part in a particular manner. For example, the wearer can be instructed to turn his or her head by approximately 45° to the right (e.g., “turn your head so your nose is pointing 45° to the right”). A synthesized 3-D virtual audio target can be generated at the specified location relative to the wearer's current head position. In response, the wearer moves his or her head in the specified direction indicated by the audio target.

In some embodiments, the exercise movements can include rotation or movement of the head while maintaining a fixed gaze. For example, the steps in Table 1 can be followed.

TABLE 1

| STEP # | DESCRIPTION |
|---|---|
| STEP 1 | Focus your eyes on the target in front of you and turn your head |
| | to the left by at least 45 degrees. |
| STEP 2 | Focus your eyes on the target in front of you and turn your head |
| | to the right by at least 45 degrees. |
| STEP 3 | Focus your eyes on the target in front of you and tip your head |
| | down by at least 30 degrees. |
| STEP 4 | Focus your eyes on the target in front of you and tip your head |
| | up by at least 30 degrees. |

These exercises can be repeated in multiple sets throughout each day or as otherwise specified by a care provider.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A method of providing vestibular therapy to a subject comprising:
prompting the subject to execute an exercise, the exercise comprising a predetermined movement while maintaining a fixed point of eye gaze;
tracking the point of gaze of the subject's eyes;
tracking movement of the subject using an IMU disposed in a fixed position relative to their head; and
generating data representing a measured deviation between the fixed point of eye gaze and the tracked point of gaze;
wherein the predetermined movement comprises movement of the head.

2-17. (canceled)

18. The method of claim 1, further comprising providing auditory guidance to the subject during the exercise.

19. (canceled)

20. The method of claim 1, further comprising detecting performance of the exercise by evaluating data from at least one of a camera and an IMU.

21. The method of claim 20, further comprising evaluating external camera data for evidence of pupil dilation or nystagmus after performance of the exercise is first detected and prompting the subject to cease performing the exercise.

22-27. (canceled)

28. The method of claim 1, further comprising tracking smoothness of movement of at least one eye when the exercise includes rotation of the front of the head while maintaining a fixed gaze.

29. The method of claim 1, wherein prompting the subject to execute an exercise comprising providing prompts according to a predetermined schedule input by a care provider.

30. The method of claim 29, further comprising changing the predetermined schedule based on at least one of the accuracy of exercise performance and the frequency of exercise performance.

31. The method of claim 1, further comprising sending information regarding at least one of the accuracy of exercise performance and the frequency of exercise performance back to the care provider.

32. The method of claim 1, wherein prompting the subject comprises queuing the prompt according to a predetermined schedule and triggering the prompt after detecting sedentary behavior of the subject.

33-34. (canceled)

35. The method of claim 1, further comprising notifying a remote care provider if nystagmus is detected in the subject.

36. The method of claim 1, wherein prompting the subject to execute an exercise comprises receiving a prompt from a remote location.

37-39. (canceled)

40. The method of claim 1, further comprising
detecting performance of the exercise by evaluating data from at least one of a camera and an IMU; and
awarding an electronic incentive to the subject if a threshold of exercise performance is met.

41. (canceled)

42. A hearing assistance device comprising:
a control circuit;
an IMU in electrical communication with the control circuit, wherein the IMU is disposed in a fixed position relative to a head of a subject wearing the hearing assistance device;
a microphone in electrical communication with the control circuit;
an electroacoustic transducer for generating sound in electrical communication with the control circuit;
a power supply circuit in electrical communication with the control circuit;
wherein the control circuit is configured to
initiate a prompt to a subject to execute an exercise, the exercise comprising a predetermined movement while maintaining a fixed point of eye gaze;
detect execution of the exercise using data derived from the IMU.

43. The hearing assistance device of claim 42, the control circuit further configured to
track the point of gaze of the subject's eyes using data received from an external camera; and
generate data representing a measured deviation between the fixed point of eye gaze and the tracked point of gaze.

44. The hearing assistance device of claim 43, further configured to provide at least one of visual and auditory feedback to the subject based on the measured deviation.

45. The hearing assistance device of claim 43, further configured to generate a score based on the measured deviation.

46. The hearing assistance device of claim 43, further configured to send information regarding the measured deviation to a remote system user.

47. The hearing assistance device of claim 43, further configured to send information regarding at least one of the accuracy of exercise performance and the frequency of exercise performance back to the care provider.

48. A system for providing vestibular training for a subject comprising
a control circuit;
an IMU in electrical communication with the control circuit;
a hearing assistance device comprising:
a microphone in communication with the control circuit;
an electroacoustic transducer for generating sound in electrical communication with the control circuit;
a power supply circuit in communication with the control circuit;
an external visual display device in wireless data communication with the hearing assistance device, the external visual display device comprising
a video display screen; and
a camera;
wherein the system is configured to
prompt the subject to execute an exercise, the exercise comprising a predetermined movement while maintaining a fixed point of eye gaze.

49. The system of claim 48, wherein the IMU is disposed in a fixed position relative to a head of a subject wearing the hearing assistance device; the system further configured to detect execution of the exercise using data derived from the IMU.

50. The system of claim 49, wherein the system is further configured to track the point of gaze of the subject's eyes using data from the camera; and
generate data representing a measured deviation between the fixed point of eye gaze and the tracked point of gaze.
